(19) United States
(12) Patent Application Publication
(10) Pub. No.: US 2008/0257356 A1
(43) Pub. Date: Oct. 23, 2008

(54) CONTROL TIP FOR SUPRAGLOTTIC AIRWAY DEVICE

(75) Inventor: John T. Swick, Hummelstown, PA (US)

Correspondence Address: GIFFORD, KRASS, SPRINKLE,ANDERSON & CITKOWSKI, P.C, PO BOX 7021, TROY, MI, 48007-7021, US

(73) Assignee: The Penn State Research Foundation, University Park, PA (US)

(21) Appl. No.: 12/107,867

(22) Filed: Apr. 23, 2008

Related U.S. Application Data
(60) Provisional application No. 60/925,806, filed on Apr. 23, 2007.

(51) Int. Cl. A61M 16/00

(52) U.S. Cl. 128/207.14

(57) ABSTRACT

An airway device for a patient is provided. The airway device can include an airway tube having a proximal end and a distal end, and a mask connected to and in fluid communication with the distal end of the airway tube. The mask has a flexible tip portion spaced apart from the distal end of the airway tube and is also configured to be disposed in sealing communication with a laryngeal inlet of the patient's airway. Also included is a control device that has a distal end attached to the flexible tip portion of the mask and a proximal end that is spaced apart therefrom. The control device is operable to flex the flexible tip portion of the mask in a direction towards the proximal end of the airway tube and thereby prevent said portion from folding back on itself in a direction away from the laryngeal inlet.

### CROSS-REFERENCE TO RELATED APPLICATIONS

This application claims priority of U.S. Provisional Patent Application Ser. No. 60/925,806 filed Apr. 23, 2007, which is incorporated herein by reference.

### FIELD OF THE INVENTION

The present invention is directed to an airway device, and more particularly to a supraglottic airway device.

### BACKGROUND OF THE INVENTION

During some medical procedures, an airway device can be used to maintain a patent airway for the patient. The airway device may be used to prevent the patient's tongue from blocking the airway and/or to ventilate the patient's lungs. Airway devices generally fall into two categories. One category of devices includes infraglottic or subglottic devices that extend below the patient's voice cords. The other category is supraglottic devices that are positioned above the patient's voice cords.

There are several designs for supraglottic airway devices. A common characteristic is that the devices must be inserted into the patient's mouth, past the tongue, through the pharynx and positioned in the larynx so as to form a seal over the patient's laryngeal inlet. One popular supraglottic device is a laryngeal mask airway (LMA). The LMA device comes in a variety of configurations, with one configuration being illustrated in FIG. 1 at reference numeral 10. As shown in this figure, the LMA device has an airway tube 20, the tube 20 having a proximal end 22 and a distal end 24. Attached to the distal end 24 is a mask 30 that can be inserted into the patient's larynx with the airway tube 20 extending from the mask 30 out of the patient's mouth. The mask 30 can include an inflatable cuff 32 that can be inflated and deflated by adding or removing air through an inflation line 40. The cuff 32 is inflated after being inserted into position so as to seal the mask 30 against the laryngeal inlet of the patient.

FIG. 2 illustrates a portion of the LMA device 10 in relation to a laryngeal anatomy of the patient. When properly inserted, the tip of the mask 30 passes beyond the soft palate, the posterior third of the tongue, the epiglottis and the laryngeal inlet 50, coming to rest on the mucus membrane 44 covering the cricoid cartilage and the upper esophagus 46. As such the mask forms a seal with the patient's laryngeal inlet 50 and provides an airway to the lungs via the trachea 48.

Turning to FIG. 3, a side view illustration of the proper placement of the mask 30 relative to the laryngeal inlet 50 is shown. However, in some instances a forward tip portion of the mask 30 can catch upon the posterior wall of the pharynx and fold back on itself in a direction away from the laryngeal inlet 50 as shown in FIG. 4. In this event, improper insertion results, a proper seal of the mask 30 with the laryngeal inlet 50 is prevented and inadequate air supply to the lungs can result. As such, an improved airway device that prevents a forward tip portion from folding back on itself during insertion would be desirable.

### SUMMARY OF THE INVENTION

An airway device for a patient is provided. The airway device can include an airway tube having a proximal end and a distal end, and a mask connected to and in fluid communication with the distal end of the airway tube. The mask has a flexible tip portion spaced apart from the distal end of the airway tube and is also configured to be disposed in sealing communication with a laryngeal inlet of the patient's airway. Also included is a control device that has a distal end attached to the flexible tip portion of the mask and a proximal end that is spaced apart therefrom. The control device is operable to flex the flexible tip portion of the mask in a direction towards the proximal end of the airway tube and thereby prevent said portion from folding back on itself in a direction away from the laryngeal inlet.

The control device can extend at least partially through a hollow channel, the hollow channel extending along at least part of a length of the airway tube. In some instances, the hollow channel is located at least partially along an outer periphery of the airway tube, at least partially along an inner periphery of the airway tube or at least partially within a sidewall of the airway tube. The control device can be a cord, wire, filament or the like with a distal end that is fixedly attached to the flexible tip portion of the mask and operable to pull the flexible tip portion in a direction towards the proximal end of the airway tube. The control device can have a proximal end that has a grasping device which is operable to be grasped by an individual. In some instances, the grasping device is a ring. In addition, the mask can have an annular peripheral portion that is made from flexible soft material and has a shape that is operable to seal against the laryngeal inlet of the patient.

### BRIEF DESCRIPTION OF THE DRAWINGS

FIG. 1 is a perspective view of a prior art embodiment of a laryngeal mask airway;

FIG. 2 is a schematic representation of a laryngeal mask airway in relation to a pharyngeal anatomy of a patient;

FIG. 3 is a side view of a proper insertion of a laryngeal mask airway within a patient;

FIG. 4 is a side view of an improper insertion of a laryngeal mask airway within a patient;

FIG. 5 is a perspective view of an embodiment of the present invention;

FIG. 6 is a perspective view of the embodiment shown in FIG. 5 illustrating a flexible tip portion of a mask being flexed by a control device;

FIG. 7 is a cross-sectional view of an airway tube for an embodiment of the present invention;

FIG. 8 is a cross-sectional view of an airway tube for an embodiment of the present invention;

FIG. 9 is a cross-sectional view of an airway tube for an embodiment of the present invention; and

FIG. 10 is a perspective view of another embodiment of the present invention.

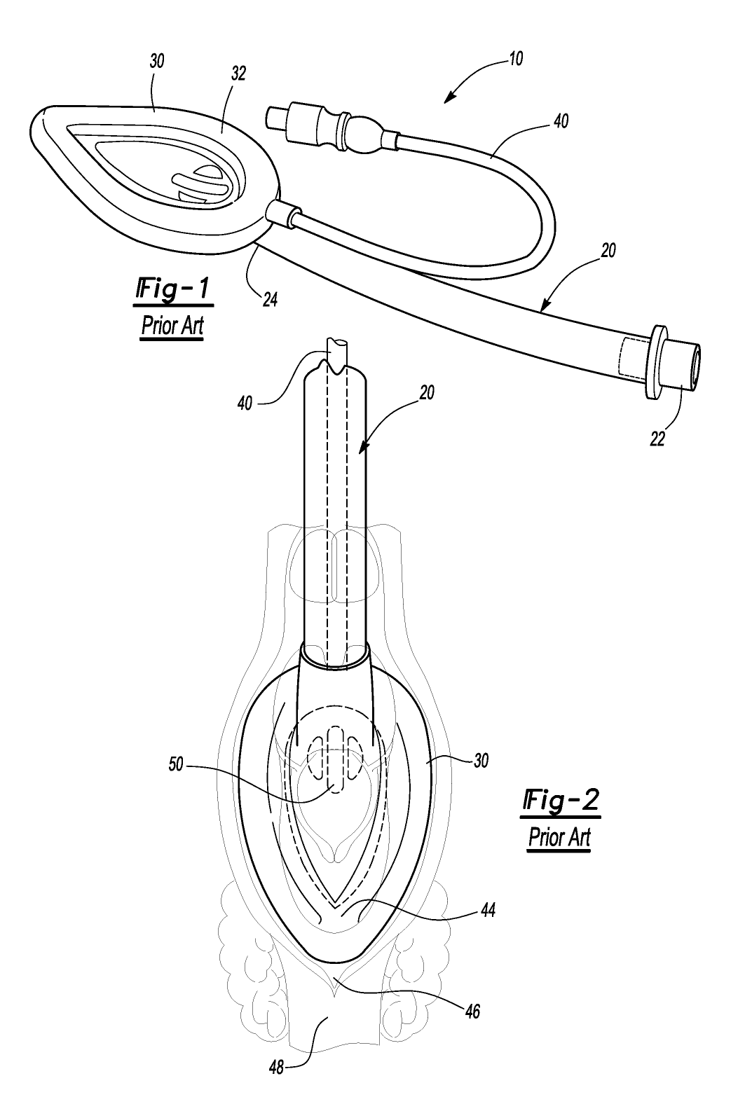

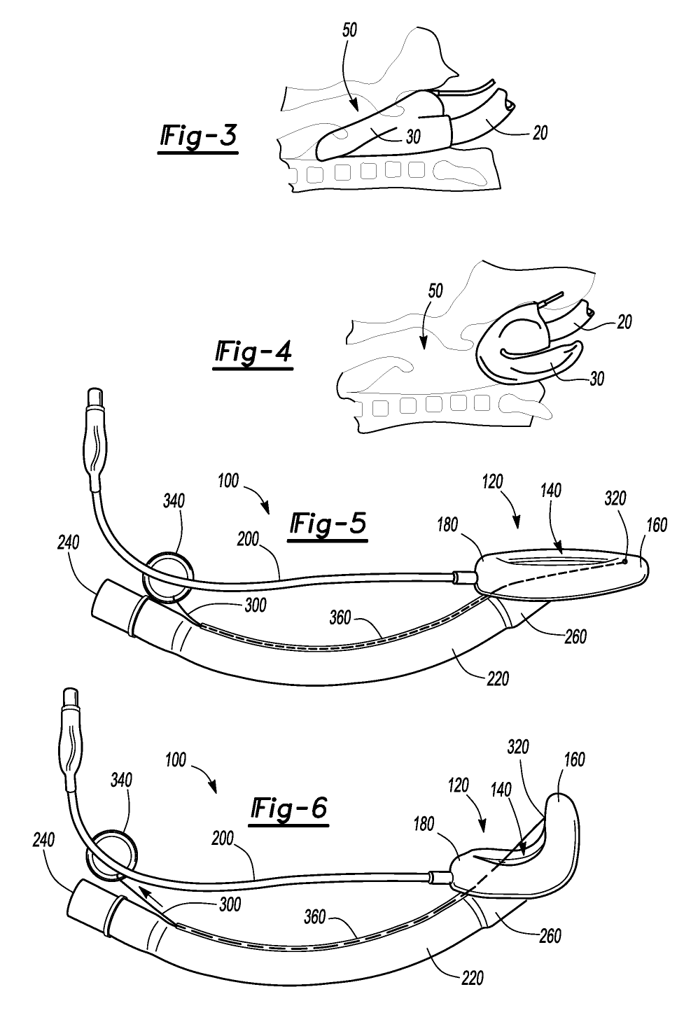

### DETAILED DESCRIPTION OF THE PREFERRED EMBODIMENTS

The present invention provides an improvement to supraglottic airway devices wherein a flexible tip portion of an airway device may be controlled during insertion. The teachings of the present invention will be illustrated with respect to a modified laryngeal mask airway (LMA). However, as will be clear to those of skill in the art, the teachings of the present invention may be applied to other supraglottic airway devices. As such, the present invention is not limited to the illustrated LMA device.

The airway device can include an airway tube having a proximal end and a distal end, and a mask connected to and in fluid communication with the distal end of the airway tube. The mask has a flexible tip portion spaced apart from the distal end of the airway tube and is also configured to be disposed in sealing communication with a laryngeal inlet of a patient's airway. Also included is a control device that has a distal end attached to the flexible tip portion of the mask and a proximal end that is spaced apart therefrom. The control device is operable to flex the flexible tip portion of the mask in a direction towards the proximal end of the airway tube and thereby prevent the tip portion from folding back on itself in a direction away from the patient's laryngeal inlet.

The control device can extend at least partially through a hollow channel, the hollow channel extending along at least part of a length of the airway tube. In some instances, the hollow channel is located at least partially along an outer periphery of the airway tube, at least partially along an inner periphery of the airway tube or at least partially within a sidewall of the airway tube. The control device can be a cord, wire, filament or the like with a distal end that is fixedly attached to the flexible tip portion of the mask and operable to pull the flexible tip portion in a direction towards the proximal end of the airway tube. The control device can have a proximal end that has a grasping device which is operable to be grasped by an individual. In some instances, the grasping device is a ring. In addition, the mask can have an annular peripheral portion that is made from flexible soft material and has a shape that is operable to seal against the laryngeal inlet of the patient.

During use, an individual takes the airway device and inserts the mask into the mouth of the patient and down into the pharynx. As the mask starts to come into contact with or approaches the posterior wall of the pharynx, the individual grasps the proximal end of the control element and by pulling thereon affords for the flexible tip portion to flex towards the proximal end of the airway tube and prevents the flexible tip portion from folding back on itself in a direction away from the patient's laryngeal inlet. Thereafter, the mask can be placed in pro per position relative to the laryngeal inlet and the airway device used to provide oxygen to the patient's lungs.

Turning now to FIG. 5, an LMA device modified in accordance with an embodiment of the present invention is illustrated. The overall device is generally indicated at reference numeral 100 and includes a mask 120 at a distal end 260 of an airway tube 220. The mask 120 includes a cuff 140 in the form of an annual peripheral portion with a distal flexible tip portion 160 and a base or upper border 180. An inflation line 200 may be used to add or remove air from the inflatable cuff 140. The airway tube 220 has a proximal end 240 defining an airway connector with the distal end 260 connected to the mask 120. The airway tube 220 may be rigid, semi-rigid or flexible and may have different shapes than shown. In addition, the mask 120 can have an aperture (not shown) that is in alignment with the distal end 260, thereby providing fluid communication between the airway tube 220 and the mask 120.

A control device is provided for controlling the position of the flexible tip portion 160 of the cuff 140. The control device includes a control element 300 such as a wire, filament, cord or the like that has a distal end 320 attached or interconnected with the tip portion 160 of the cuff 140 and a proximal end 340. The proximal end 340 can include a grasping device, illustratively including a ring, the grasping device operable to be grasped by an individual and used to pull upon the control element 300 and flex the tip portion 160. A guide, housing or hollow channel 360 can be provided and the control element 300 can extend at least partially therethrough. In some instances, the guide 360 extends along at least part of a length of the tube 220 and can be located at least partially along an outer periphery of the airway tube 220, at least partially along an inner periphery of the airway tube 220 or at least partially within a sidewall of the airway tube 220.

Turning now to FIG. 6 where like numerals correspond to like elements referenced in previous figures, when the proximal end 340 is pulled, the tip portion 160 being attached to the control element 300 moves towards the base 180 of the cuff 140. During use, an individual can pull on the proximal end 340 when the tip portion 160 approaches or encounters the posterior wall of the pharynx and thereby curve or fold the cuff 140 as shown in the figure. In this manner, the control element aids the LMA device to “turn the corner” into the pharynx. The proximal end 340 can then be released and the device 100 used in a traditional manner.

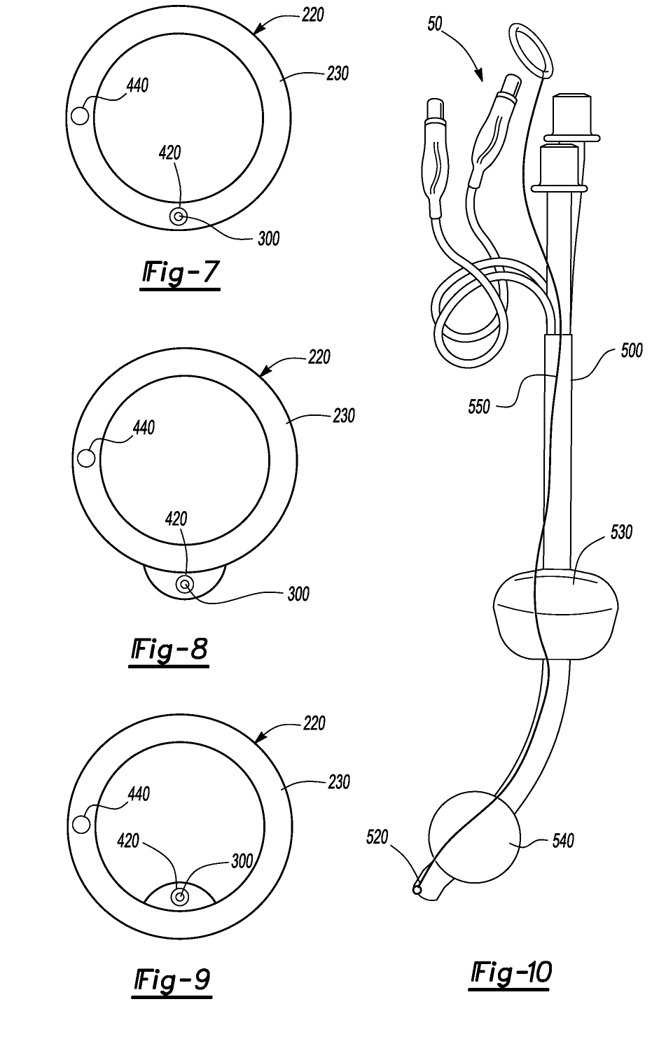

FIGS. 7-9 provide cross-sectional views of the airway tube 220 according to various embodiments of the present invention. The airway tube 220 has a sidewall 230 with the control element 300 extending through a hollow channel 420 defined in the sidewall 230, as in FIG. 7, on the outer periphery of the tube 220 as shown in FIG. 8 or on the inner periphery of the tube 220 as shown in FIG. 9. Other positions and configurations may also be used, as will be clear to those of skill in the art. In some instances, the inflation line 200 may be replaced with an inflation channel 440 defined in the sidewall 230 of the tube 220, as shown in FIGS. 7-9.

FIG. 10 illustrates another type of supraglottic airway device known as a Combi Tube shown generally at reference numeral 50. The Combi Tube 50 includes an airway tube 500 with an upper inflatable collar 530 and a lower inflatable collar 540. When properly inserted through the mouth of the patient, the lower inflatable cuff 540 is positioned below the laryngeal inlet and the upper inflatable cuff 530 is positioned above the laryngeal inlet. Both cuffs are then inflated so as to seal a passage to the laryngeal inlet. According to one embodiment, the Combi Tube 50 may be modified such that a distal end 520 of the airway tube 500 is attached with a control element 550 such that the distal end 520 can be flexed and controlled by a surgeon inserting the device. It is appreciated that FIG. 10 illustrates the control element 550 not being located at least partially within a hollow channel, however the control element 550 can optionally be located within a hollow channel as taught in FIGS. 5-9 and the description above. Similar modifications can be made to other supraglottic devices.

As will be clear to those of skill in the art, the embodiments of the present invention illustrated herein may be modified in additional ways without departing from the scope or teaching of the present invention. It is the following claims, including all equivalents, which define the scope of the present invention.

I claim:

1. An airway device for a patient, said airway device comprising:
an airway tube having a proximal end and a distal end;
a mask connected to and in fluid communication with said distal end of said airway tube, said mask having a flexible tip portion spaced apart from said distal end of said airway tube, said mask also configured to be disposed in sealing communication with a laryngeal inlet of the patient airway; and
a control device having a distal end attached to said flexible tip of said mask and a proximal end spaced apart therefrom, said control device operable to flex said flexible tip in a direction towards said proximal end of said airway tube.

2. The airway device of claim 1, wherein said control device extends at least partially through a hollow channel, said hollow channel extending along at least part of a length of said airway tube.

3. The airway device of claim 2, wherein said hollow channel is located at least partially along an outer periphery of said airway tube.

4. The airway device of claim 2, wherein said hollow channel is located at least partially along an inner periphery of said airway tube.

5. The airway device of claim 2, wherein said hollow channel is located at least partially within a sidewall of said airway tube.

6. The airway device of claim 1, wherein said control device is a cord, said distal end of said cord fixedly attached to said flexible tip of said mask and said cord operable to pull said flexible tip of said mask in a direction towards said proximal end of said airway tube.

7. The airway device of claim 6, wherein said proximal end of said cord has a grasping device operable to be grasped by an individual.

8. The airway device of claim 7, wherein said grasping device is a ring.

9. The airway device of claim 1, wherein said mask has an annular peripheral portion, said annular peripheral portion made of a flexible soft material and having a shape operable to seal against the laryngeal inlet of the patient.

10. The airway device of claim 9, wherein said annular peripheral portion of said mask is inflatable.

11. A supraglottic airway device for a patient, said airway device comprising:
an airway tube having a proximal end and a distal end;
a mask attached to said distal end of said airway tube and configured to be disposed in sealing communication with a laryngeal inlet of the patient airway, said mask having an aperture in alignment with and a flexible tip portion spaced apart from said distal end of said airway tube; and
a cord having a distal end attached to said flexible tip of said mask and a proximal end spaced apart therefrom, said cord operable to be pulled upon and to flex said flexible tip in a direction towards said proximal end of said airway tube and prevent said flexible tip from folding back on itself in a direction away from the laryngeal inlet of the patient.

12. The airway device of claim 11, wherein said cord extends at least partially through a hollow channel, said hollow channel extending along at least part of a length of said airway tube.

13. The airway device of claim 12, wherein said hollow channel is located at least partially along an outer periphery of said airway tube.

14. The airway device of claim 12, wherein said hollow channel is located at least partially along an inner periphery of said airway tube.

15. The airway device of claim 12, wherein said hollow channel is located at least partially within a sidewall of said airway tube.

16. The airway device of claim 11, wherein said proximal end of said cord has a grasping device operable to be grasped by an individual.

17. The airway device of claim 11, wherein said mask has an inflatable annular peripheral portion, said annular peripheral portion made of a flexible soft material and having a shape operable to seal against the laryngeal inlet of the patient.

18. A supraglottic airway device for a patient, said airway device comprising:
an airway tube having a proximal end and a distal end;
a hollow channel attached to at least part of said airway tube;
a mask connected to and in fluid communication with said distal end of said airway tube, said mask having a flexible tip portion spaced beyond said distal end of said airway tube, said mask also having an inflatable annular peripheral portion configured to be disposed in sealing communication with a laryngeal inlet of the patient airway;
an inflation tube in fluid communication with said inflatable annular peripheral portion of said mask; and
a cord extending at least partially through said hollow channel, said cord having a distal end attached to said flexible tip of said mask and a grasping end located proximate said proximal end of said airway tube, said cord operable to be grasped at said grasping end and pulled such that said flexible tip is flexed in a direction towards said proximal end of said airway tube and prevented from folding back on itself in a direction away from the laryngeal inlet of the patient.

19. The airway device of claim 18, wherein said hollow channel is located at a position selected from the group consisting of at least partially along an outer periphery of said airway tube, at least partially along an inner periphery of said airway tube and at least partially within a sidewall of said airway tube.

20. The airway device of claim 18, wherein said grasping end of said cord has a ring attached to said cord.
